(19) United States
(12) Patent Application Publication
(10) Pub. No.: US 2023/0346559 A1
(43) Pub. Date: Nov. 2, 2023

(54) ANNULOPLASTY RING WITH FLEXIBLE OR BREAKABLE JOINT

(71) Applicant: EDWARDS LIFESCIENCES CORPORATION, Irvine, CA (US)

(72) Inventors: Roland Patrick Moder, Fountain Valley, CA (US); Lynne Borowski Shwed, Park City, UT (US)

(21) Appl. No.: 18/348,976

(22) Filed: Jul. 7, 2023

Related U.S. Application Data
(63) Continuation of application No. PCT/US2022/011554, filed on Jan. 7, 2022.
(60) Provisional application No. 63/134,932, filed on Jan. 7, 2021.

(51) Int. Cl. A61F 2/24

(52) U.S. Cl. CPC A61F 2/2466; A61F 2/2454; A61F 2/2448

(57) ABSTRACT

A device for treating a heart valve comprises a first body portion, a second body portion, and a first deformable portion situated between the first body portion and the second body portion. The first body portion, the second body portion, and the first deformable portion are configured to be attached to an annulus portion of the heart valve.

### CROSS REFERENCE TO RELATED APPLICATIONS

This application is a continuation of International Patent Application No. PCT/US2022/011554, filed Jan. 7, 2022, which claims the benefit of U.S. Patent Application No. 63/134,932, filed on Jan. 7, 2021, the entire disclosures all of which are incorporated by reference for all purposes.

### BACKGROUND

### Field

The present disclosure relates to the field of medical devices and procedures.

### Description of the Related Art

An annuloplasty is a procedure to tighten or reinforce the ring (annulus) around a valve in the heart. For example, due to various factors, two or more leaflets of a heart valve may not coapt properly, resulting in regurgitation of the blood flow (e.g., backwards blood flow). An annuloplasty ring may be attached (e.g., sewn) to the annulus of the heart valve to pull the leaflets together for proper coaptation and to re-establish proper valve function.

### SUMMARY

Some implementations of the present disclosure relate to a device for treating a heart valve. The device comprises a first body portion, a second body portion, and a first deformable portion situated between the first body portion and the second body portion. The first body portion, the second body portion, and the first deformable portion are configured to be attached to an annulus portion of the heart valve.

The device may further comprise an outer covering configured to surround the first body portion, the second body portion, and the first deformable portion. In some embodiments, the first deformable portion is configured to break in response to expansion forces.

In some embodiments, the first deformable portion is configured to bend in response to expansion forces. The first body portion, the second body portion, and the first deformable portion may be configured to form a partial ring with a gap between the first body portion and the second body portion.

The first body portion, the second body portion, and the first deformable portion may be configured to form a continuous ring. In some embodiments, the device further comprises a second deformable portion situated between the first body portion and the second body portion and across from the first deformable portion.

In some embodiments, the first body portion, the second body portion, the first deformable portion, and the second deformable portion are configured to form a continuous ring in a generally circular shape. The first body portion, the second body portion, the first deformable portion, and the second deformable portion may be configured to form a continuous ring having a first generally flat portion.

The first deformable portion may be situated at the first generally flat portion. In some embodiments, the device further comprises a third body portion, a second deformable portion situated between the second body portion and the third body portion, and a third deformable portion situated between the first body portion and the third body portion.

Some implementations of the present disclosure relate to a method for treating a heart valve. The method comprises delivering a deformable device to an annulus of the heart valve. The deformable device comprises a first body portion, a second body portion, and a first deformable portion situated between the first body portion and the second body portion. The first body portion, the second body portion, and the first deformable portion are configured to be attached to an annulus portion of the heart valve. The method further comprises delivering a subsequent repair device to the heart valve, permanently deforming the deformable device to allow attachment of the subsequent repair device at the heart valve, and attaching the subsequent repair device at the heart valve.

In some embodiments, the subsequent repair device is a replacement heart valve. The deformable device may further comprise an outer covering configured to surround the first body portion, the second body portion, and the first deformable portion.

The first deformable portion may be configured to break in response to expansion forces from a medical device. In some embodiments, the first deformable portion is configured to bend in response to expansion forces from a medical device.

In some embodiments, the first body portion, the second body portion, and the first deformable portion are configured to form a non-continuous ring with a gap between the first body portion and the second body portion. The first body portion, the second body portion, and the first deformable portion may be configured to form a continuous ring.

The method may further comprise a second deformable portion situated between the first body portion and the second body portion and across from the first deformable portion. In some embodiments, the deformable device further comprises a third body portion, a second deformable portion situated between the second body portion and the third body portion, and a third deformable portion situated between the first body portion and the third body portion.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the disclosure. In addition, various features of different disclosed examples can be combined to form additional examples, which are part of this disclosure. Throughout the drawings, reference numbers may be reused to indicate correspondence between reference elements.

FIG. 1 illustrates an example representation of a heart having various features relevant to certain embodiments of the present inventive disclosure.

FIG. 2A provides a cross-sectional view of the left ventricle and left atrium of an example heart.

FIG. 2B illustrates a top/surgeon's view looking at the mitral valve of the heart.

FIG. 3A provides an illustration of the heart in a state where functional mitral valve regurgitation (FMR) is present.

FIG. 3B show a mitral valve where the annulus is dilated and deformed causing mitral regurgitation.

FIG. 4 illustrates an example mitral valve with an annuloplasty ring implanted thereon in an attempt to restore proper function of the mitral valve in accordance with one or more examples.

FIG. 5 illustrates an example tricuspid valve 8 with an annuloplasty ring implanted thereon in an attempt to restore proper function of the mitral valve in accordance with one or more examples.

FIGS. 6A and 6B illustrate an example implant in accordance with some embodiments of the present disclosure.

FIGS. 7A-7C illustrate another example implant in accordance with one or more embodiments of the present disclosure.

FIGS. 8A-8C illustrate another example implant in accordance with one or more embodiments of the present disclosure.

FIGS. 9A and 9B illustrate example implants comprising multiple deformable portions, in accordance with one or more embodiments of the present disclosure.

FIG. 10 is a flowchart illustrating steps of a process for delivering and/or deforming a valve repair device in accordance with one or more embodiments of the present disclosure.

FIG. 11 provides certain images depicting various features associated with steps of the process depicted in FIG. 10.

### DETAILED DESCRIPTION

The headings provided herein are for convenience only and do not necessarily affect the scope or meaning of the claimed subject matter. The present disclosure relates to systems, devices, and methods to determine access for an anatomical feature based on an analysis of one or more images representing a mineral deposit.

Although certain examples are disclosed below, the subject matter extends beyond the specifically disclosed examples to other alternative examples and/or uses, and to modifications and equivalents thereof. Thus, the scope of the claims that may arise here from is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Overview

As noted above, an annuloplasty procedure can be performed to tighten or reinforce the ring (annulus) around a valve in the heart. Such procedures involve attaching a structure (e.g., annuloplasty ring) to the annulus of the heart valve. Various types of annuloplasty rings have been developed to satisfy the myriad of contexts in which an annuloplasty ring may be implanted (e.g., different sized heart valves, heart valve abnormalities, physician preferences, etc.). In particular, annuloplasty rings come in different sizes, shapes, materials, suture features for attachment, and so on, which provide physicians with options to implement. In many cases, a physician can use one or more ring sizers to determine a size of an annuloplasty ring to use. The physician can overlay D-shaped plates (e.g., the ring sizers) of different sizes onto the heart valve to identify an optimal size of an annuloplasty ring for the specific heart valve.

In some cases, annuloplasty rings may be replaced and/or supplemented in subsequent procedures by additional implants. For example an annuloplasty ring may be replaced and/or supplemented with a replacement valve implant (e.g., during a transcatheter aortic valve replacement (TAVR) procedure). Some annuloplasty rings may not be designed to be replaced and/or supplemented. For example, some annuloplasty rings may be composed of generally elastic materials that may to return to a formed shape after any applied stresses are relieved and thus may not be mechanically deformed in a significant way (e.g., with a balloon and/or balloon expandable valve). Some annuloplasty rings can provide a very rigid constraint to the annulus that can prevent replacement valves from deploying in an optimal shape (e.g., a parallel shape).

This disclosure describes techniques related to annuloplasty rings having features configured to facilitate placement of subsequently delivered devices at or near the annuloplasty rings. In some embodiments, an annuloplasty ring may comprise one or more segments/body portions having and/or connected by a deformable (e.g., breakable and/or flexible) portion, which may include any means for deforming an implant. The deformable portion may be permanently deformable and/or may be at least partially over-molded with a covering. Some annuloplasty rings may have a continuous (e.g., a “D-shaped structure) and/or non-continuous structure (e.g., a “C-shaped” structure) that may allow for future implanted devices to be able to expand the annuloplasty ring somewhat. One or more deformable portions may be configured to be situated between body portions of the implant. The body portions, which may include any means for treating, tightening, and/or reinforcing an annulus portion of a heart valve, and/or deformable portions may be configured to form a continuous and/or non-continuous implant which may be configured to be attached to an annulus portion of a heart valve. The implant may be attached through any suitable means, including using sutures and/or any other means for attaching the implant to an annulus and/or surrounding portion of a valve of a heart.

Some embodiments may utilize at least one flexible and/or breakable hinge configured to enable a balloon and/or mechanically expanded valve to permanently deform the annuloplasty ring once a threshold force/strain level is reached. This may allow a newly implanted device to be deployed to a designed/optimal shape which can result in better homodynamic performance and/or lower chances of failure due to non-uniform stresses. In some embodiments, a deformable joint may comprise a brittle (e.g., ceramic) material to allow for a balloon and/or mechanically expanded valve to break the joint once sufficient forces are generated to eliminate the additional constraint from the annuloplasty ring.

### Example Heart Anatomy

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

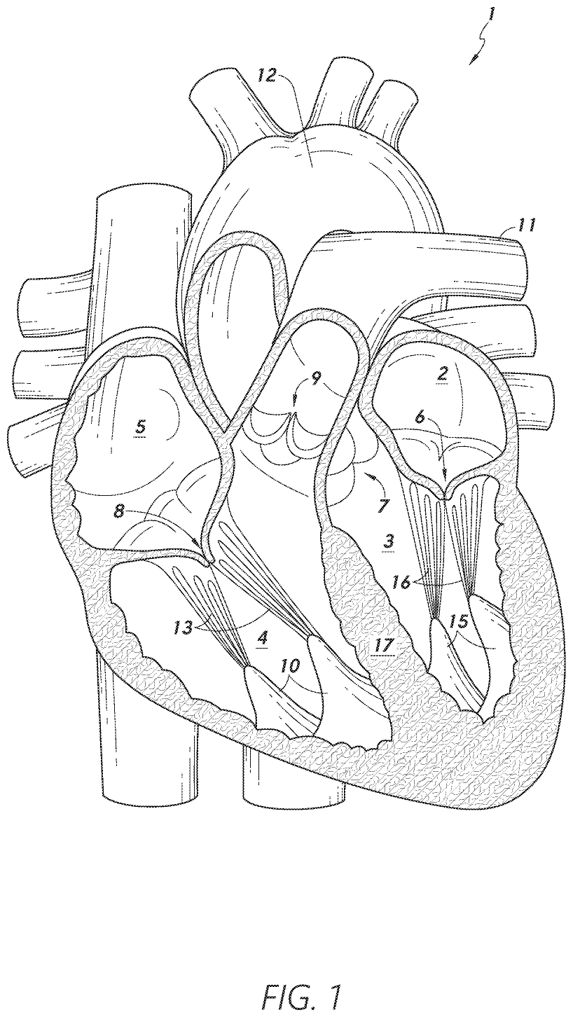

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex (or apex region) and is located on the midclavicular line, in the fifth intercostal space.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery ii, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (e.g., mitral and tricuspid) heart valves may further comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to the anterior, posterior and septal leaflets of the tricuspid valve, respectively, via the chordae tendineae 13. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

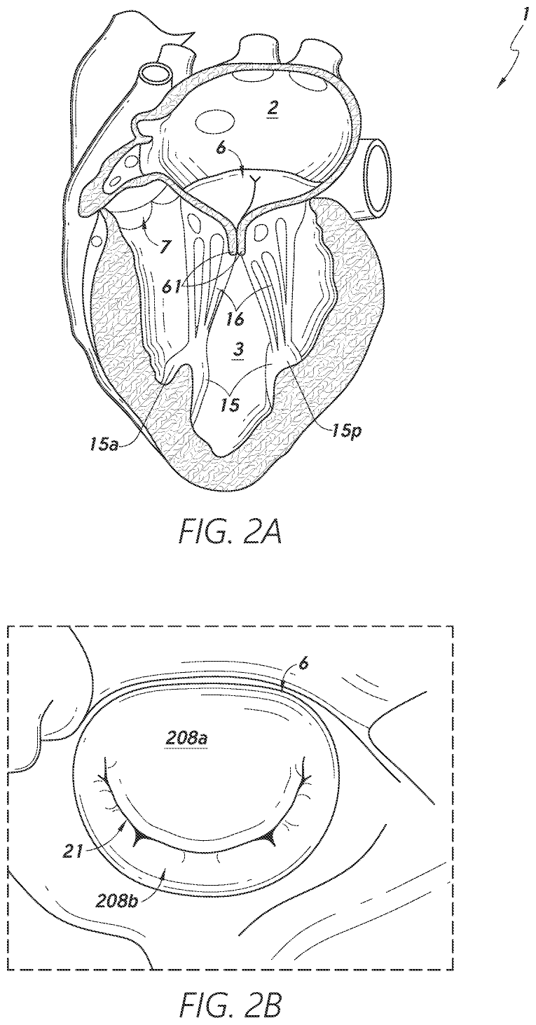

FIG. 2A provides a cross-sectional view of the left ventricle 3 and left atrium 2 of an example heart 1. While some embodiment devices and/or methods are described herein with respect to the left ventricle 3, mitral valve 6, and/or left atrium 2, such devices and/or methods may be applied to and/or performed within other areas of the heart, including the right ventricle, right atrium, and/or tricuspid valve. The diagram of FIG. 2A shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2A and various other figures provided herewith and described herein in the context of certain embodiments of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve 6 as shown in FIG. 2A, the valve leaflets may extend inward from the valve annulus and come together in the flow orifice to permit flow in the outflow direction (e.g., the downward direction in FIG. 2A) and prevent backflow or regurgitation toward the inflow direction (e.g., the upward direction in FIG. 2A). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position. The papillary muscles 15 may include a first papillary muscle 15a (e.g., an anterolateral papillary muscle, which may be primarily tethered to the anterior leaflet, for example) and a second papillary muscle 15p (e.g., the posteromedial papillary muscle, which may be primarily tethered to the posterior leaflet, for example). Each of the first papillary muscle 15a and second papillary muscle 15p may provide chordae tendineae 16 to each valve leaflet (e.g., the anterior and posterior leaflets). With respect to the state of the heart 1 shown in FIG. 2A, the proper coaptation of the valve leaflets, which may be due in part to proper position of the papillary muscles 15, may advantageously result in mitral valve operation substantially free of leakage.

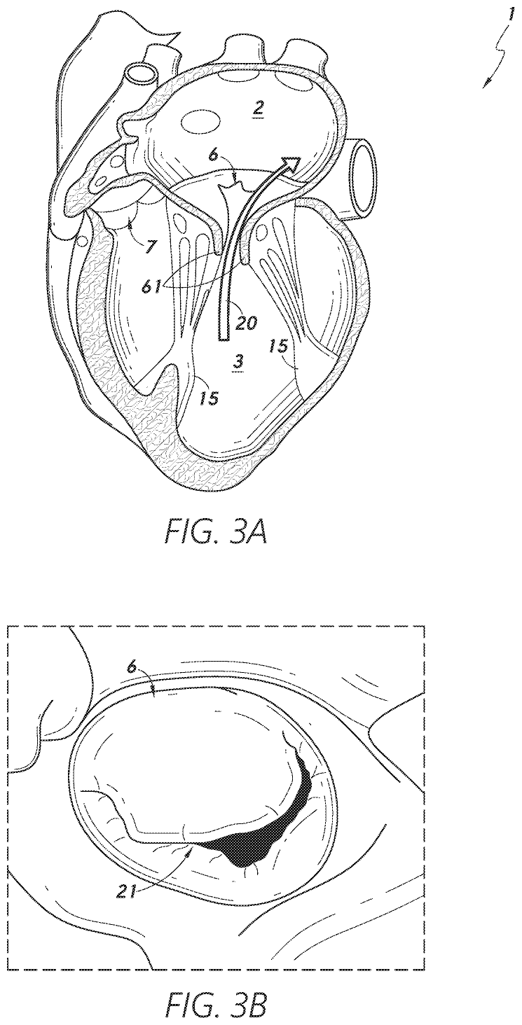

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3A, which illustrates a cross-sectional view of a heart 1 experiencing mitral regurgitation flow 20, dilation of the left ventricle may cause changes in the position of the papillary muscles 15 that allow flow 20 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

FIG. 2B illustrates a top/surgeon's view looking at the mitral valve 6 of the heart 1. The mitral valve 6 generally includes an anterior leaflet 208a that is relatively large and attaches to the anterior segment of the annulus, while a posterior leaflet 208b is smaller but extends further circumferentially and attaches to the posterior segment of the annulus, as shown in FIG. 2B. The anterior leaflets 208a and the posterior leaflets 208b join and insert into the annulus at the commissures 21, namely the anterior commissure and posterior commissure.

### Example Mitral Valve Conditions

Several diseases can affect the structure and function of the mitral valve. The mitral valve and, less frequently, the tricuspid valve, are prone to deformation and/or dilation of the valve annulus, tearing of the chordae tendineae, and/or leaflet prolapse, which results in valvular insufficiency wherein the valve does not close properly and allows for regurgitation or back flow from the left ventricle into the left atrium. Deformations in the structure or shape of the mitral or tricuspid valve can be repairable.

Mitral regurgitation is one of the most common valvular malfunctions in the adult population, and typically involves the elongation or dilation of the posterior two-thirds of the mitral valve annulus, the section corresponding to the posterior leaflet. The most common etiology of systolic mitral regurgitation is myxomatous degeneration, also termed mitral valve prolapse (29% to 70% of cases), which afflicts about 5 to 10 percent of the population in the U.S. Women are affected about twice as often as men. Myxomatous degeneration has been diagnosed as Barlow's syndrome, billowing or ballooning mitral valve, floppy mitral valve, floppy-valve syndrome, prolapsing mitral leaflet syndrome, or systolic click-murmur syndrome. The symptoms include palpitations, chest pain, syncope or dyspnea, and a mid-systolic click (with or without a late systolic murmur of mitral regurgitation). These latter symptoms are typically seen in patients with Barlow's syndrome, where extensive hooding and billowing of both leaflets are the rule. Some forms of mitral valve prolapse seem to be hereditary, though the condition has been associated with Marfan's syndrome, Grave's disease, and other disorders.

Myxomatous degeneration involves weakness in the leaflet structure, leading to thinning of the tissue and loss of coaptation. Barlow's disease is characterized by myxoid degeneration and appears early in life, often before the age of fifty. In Barlow's disease, one or both leaflets of the mitral valve protrude into the left atrium during the systolic phase of ventricular contraction. The valve leaflets are thick with considerable excess tissue, producing an undulating pattern at the free edges of the leaflets. The chordae are thickened, elongated and may be ruptured. Papillary muscles are occasionally elongated. The annulus is dilated and sometimes calcified. Some of these symptoms are present in other pathologies as well and, therefore, the present application may refer to myxoid degeneration, which is the common pathologic feature of the various diagnoses, including Barlow's syndrome.

Other causes of mitral regurgitation include ischemic heart disease with ischemic mitral regurgitation (IMR), dilated cardiomyopathy (in which the term “functional mitral regurgitation FMR is used), rheumatic valve disease, mitral annular calcification, infective endocarditis, fibroelastic deficiency (FED), congenital anomalies, endocardial fibrosis, and collagen-vascular disorders. IMR is a specific subset of FMR, but both are usually associated with morphologically normal mitral leaflets. Thus, the types of valve disease that lead to regurgitation are varied and present vastly differently.

FIG. 3A provides an illustration of the heart 1 in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIGS. 3A and 3B may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3A, the leaflets 61 of the mitral valve 6 (or tricuspid valve) are not in a state of coaptation, resulting in an opening between the mitral valve leaflets 61 during the systolic phase of the cardiac cycle, which allows the leakage flow 20 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3A) and/or unwanted backflow or regurgitation toward the inflow direction (e.g., the downward direction in FIG. 3A).

FIG. 3B show a mitral valve 6 where the annulus is dilated and deformed causing mitral regurgitation through the commissure 21. At a structural level, four general types of structural changes of the mitral valve apparatus can cause regurgitation: leaflet retraction from fibrosis and calcification, annular dilation, chordal abnormalities (including rupture, elongation, shortening, or apical tethering or “tenting” as seen in FMR and IMR), and possibly papillary muscle dysfunction.

Example Mitral Valve with Annuloplasty Ring

Various techniques/procedures may be used to repair diseased or damaged heart valves, such as mitral and tricuspid valves. These include, but are not limited to, annuloplasty (e.g., contracting the valve annulus to restore the proper size and/or shape of the valve), quadrangular resection of the leaflets (e.g., removing tissue from enlarged or misshapen leaflets), commissurotomy (e.g., cutting the valve commissures to separate the valve leaflets), shortening and transposition of the chordae tendineae, reattachment of severed chordae tendineae or papillary muscle tissue, and decalcification of valve and annulus tissue.

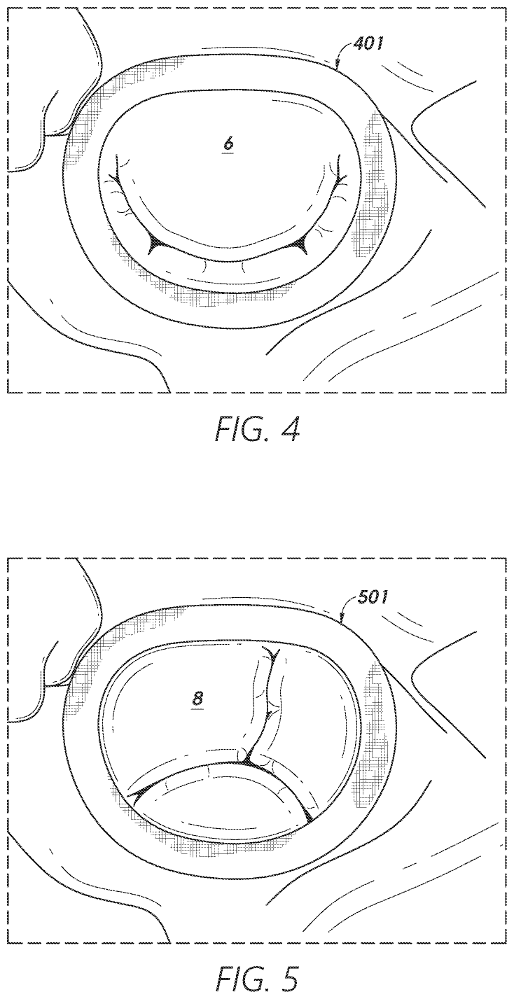

FIG. 4 illustrates an example mitral valve 6 with an annuloplasty ring 401 implanted thereon in an attempt to restore proper function of the mitral valve 6 in accordance with one or more examples. In examples, the aim of an annuloplasty ring is to restore the shape of the mitral annulus or, in some conditions, to overcorrect the shape by pulling a segment of the annulus inward. In this example, the mitral valve 6 had a deformed annulus leading to regurgitation and the annuloplasty ring 401 is implanted to restore the mitral valve 6 to the normal shape, as shown. The annuloplasty ring 401 can be representative of any of the annuloplasty rings discussed herein. The annuloplasty ring 401 can be sutured to the deformed annulus or attached in another manner. The annuloplasty ring 401 can include a covering (e.g., fabric covering) over a structural interior support or body. In some cases, a suture-permeable interface fills a space between the covering and the interior body. The annuloplasty ring 401 can have a closed or continuous periphery. Here, the annuloplasty ring 401 is illustrated with a particular form; however, the annuloplasty ring 401 can include other forms, such as other shapes, sizes, materials, and so on.

Although various techniques/procedures are discussed herein in the context of mitral valves, the techniques/procedures can be applicable to other types of heart valves and/or anatomical structures/features.

For example, FIG. 5 illustrates an example tricuspid valve 8 with an annuloplasty ring 501 implanted thereon in an attempt to restore proper function of the tricuspid valve 8 in accordance with one or more examples.

In some embodiments, annuloplasty rings and/or other implants described herein may be configured for delivery and/or attachment to an annulus portion of a heart valve to treat the heart valve. An implant may be sized slightly smaller than a distended annulus.

Various implants described herein may comprise inner body portions at least partially encapsulated and/or covered by an outer sheath and/or multiple outer sheath portions. In some embodiments, one or more body portions of an implant may comprise a relatively rigid and/or elastic inner structural support at least partially surrounded by a pliable core material and/or a cover composed of fabric and/or other materials. For example, an implant (e.g., an annuloplasty ring) may comprise an inner skeleton of one or more bands of relatively rigid and/or elastic material (e.g., Elgiloy) surrounded by a suture-permeable core material (e.g., silicone) and/or an outer fabric cover. In some embodiments, the inner skeleton may comprise multiple bands separated by plastic and/or other relatively low friction material (e.g., TEFLON) to allow the bands to more easily flex with respect to one another.

In some embodiments, an implant (e.g., an annuloplasty ring) may be configured to implanted at an annulus using any of a variety of attachment means, which can include one or more sutures. For example, one or more sutures may be distributed around body portions of the implant and/or may be tied off to present minimal surface roughness and/or to reduce the chance of thrombi forming thereon.

The inner structural support and/or pliable core material of one or more body portions of an implant may be relatively rigid and/or may be configured to initially resist deformation when subjected to the stress imparted thereon by the valve annulus of an operating human heart and/or by various medical devices (e.g., balloon expandable devices). The term “deformation” is used herein in accordance with its plain and ordinary meaning and may refer to substantial permanent deformation from a predetermined manufactured and/or shape-set shape. A number of generally rigid materials can be utilized, including various bio-compatible polymers, metals, and/or alloys. An implant may comprise one or more deformable portions which may be configured to deform in response to pressure from a medical device and/or growth of a native heart valve.

### Deformable Implants

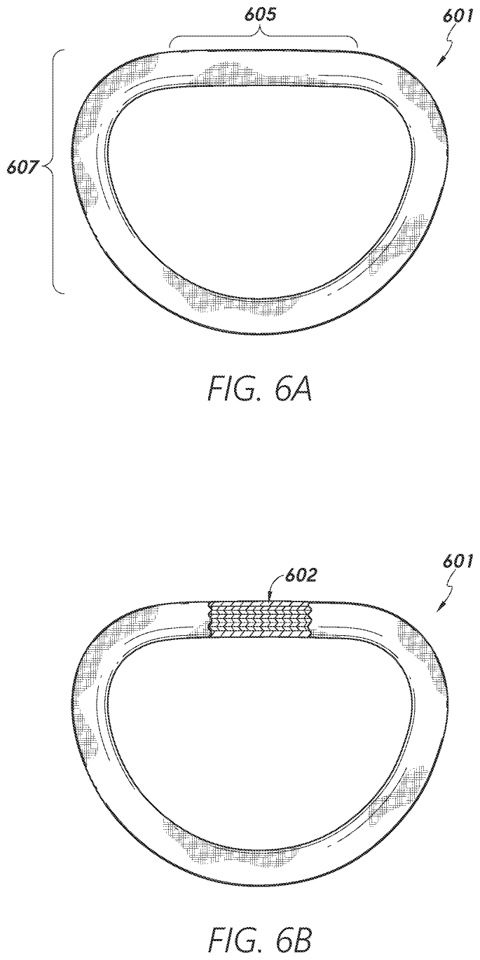

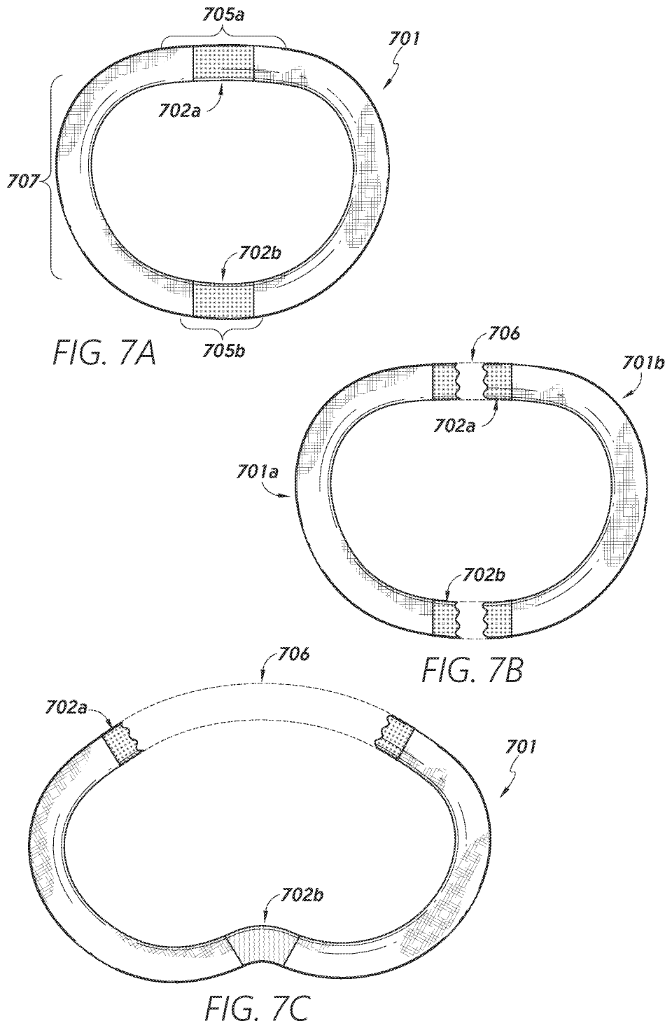

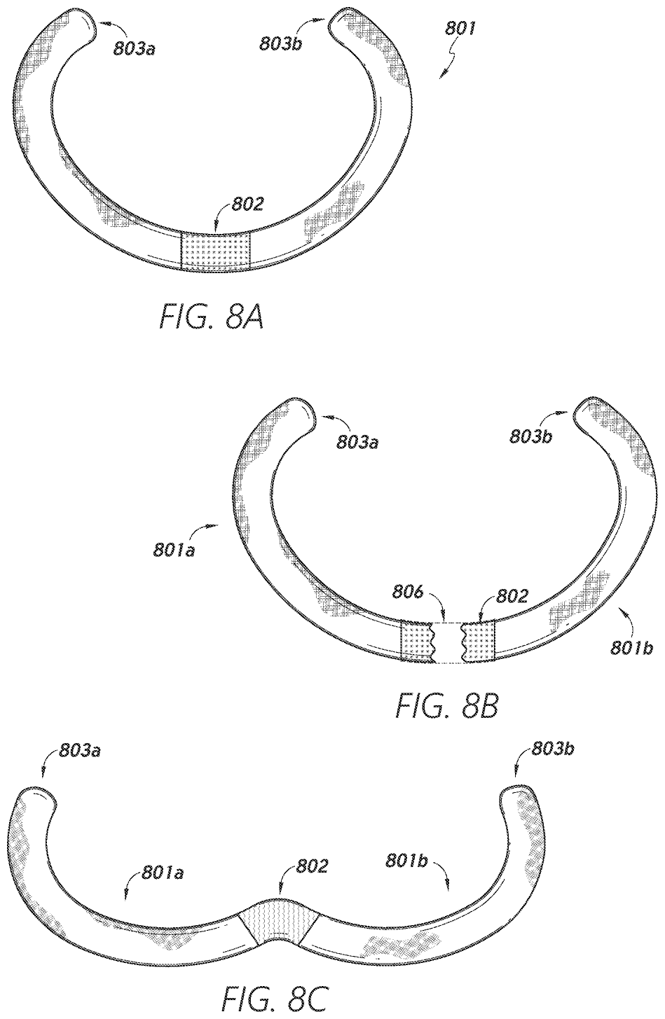

FIGS. 6A and 6B illustrate an example implant 601 (e.g., an annuloplasty ring) in accordance with some embodiments of the present disclosure. The implant 601 shown in FIGS. 6A and 6B has a “D-shaped” structure, in which the implant 601 comprises a generally flat and/or reduced curvature portion 605 which extends on either side into a generally curved portion 607 (which may comprise the entire implant 601 other than the generally flat portion 605) configured to form a continuous loop. For example, the generally flat portion 605 may have a reduced curvature relative to the curvature of the generally curved portion 607. However, the description of the implant 601 herein and/or the description of the various implants illustrated in FIGS. 6-9 may have any of a variety of structures, which may include a continuous and/or closed (e.g., circular, ovular, “D-shaped,” and/or “O-shaped”) structure (see, e.g., FIGS. 6A, 6B, 7A-7C, 9A, and/or 9B) and/or a discontinuous and/or open (e.g., “C-shaped”) structure (see, e.g., FIGS. 8A-8C). The various features described with respect to FIGS. 6-9 may be applicable to more than one possible structures and/or the particular structures shown in FIGS. 6-9 are provided for illustrative purposes and should not be understood to be limiting.

The effect of providing the generally flat portion 605 may be to remodel the valve annulus in a particular region (e.g., at the posterior leaflet scallop of the mitral valve) closer toward a flow axis than other regions (e.g., scallops) of the valve. The reduction of the anterior-posterior dimension of the annulus in this manner may more effectively correct for dysfunctions that can exist (e.g., in ischemic mitral valve insufficiency).

In some embodiments, an implant 601 may comprise various layers of materials. For example, an implant 601 may be at least partially enclosed by a covering, which may include any means for covering at least a portion of an implant. Coverings may be at least partially composed of cloth, polyurethane, and/or any other material suitable for contact with human tissue. The covering may comprise a knitted structure with optimal tension to avoid irregularities and/or wrinkles.

The implant 601 may comprise one or more deformable portions 602 as shown in FIG. 6B. For illustrative purposes, the covering is not shown over the deformable portion 602 in FIG. 6B to show an example position and/or structure of the deformable portion 602. However, a covering of the implant 601 may be configured to at least partially cover the deformable portion 602 and/or other portions of the implant 601. In some embodiments, the deformable portion 602 may be at least partially composed of one or more generally flexible (e.g., various polymeric elastomers, including polyurethane elastomer (PUE)) and/or breakable materials. Example breakable materials can include generally brittle polymers including acrylic and/or constructions and/or networks of polymer fibers (e.g., braided materials) having relatively low creep properties (e.g., ultra-high-molecular-weight polyethylene (UHMWPE)). Deformable portions 602 composed at least partially of multiple fiber structures may provide improved tensile strength due at least in part to the relatively high number of fibers in a bundle of fibers.

In some embodiments, one or more deformable portions 602 may be configured to at least partially bend in response to expansion forces at the implant 601 (e.g., created by a balloon and/or mechanical expansion device). The deformable portion 602 may be composed at least partially of non-elastic materials such that the deformable portion 602 may be configured to cause permanent deformation of the implant 601 in response to expansion forces. In this way, the implant 601 may be configured to be minimally restrictive to other devices (e.g., replacement valves) delivered during future procedures.

The deformable portion 602 may be at least partially composed of materials configured to break to create a disconnect and/or separation between some portions of the implant. In some embodiments, a covering may be configured to extend over disconnected portions of the implant 601. For example, breaking of the deformable portion 602 may not be configured to cause a break and/or tear in a covering over the deformable portion 602. In some embodiments, the covering may advantageously be configured to retain fragments and/or pieces of a broken deformable portion 602 and/or to prevent such fragments and/or pieces from contacting tissue and/or entering a blood stream.

While a single deformable portion 602 is shown in FIGS. 6A and 6B, the implant 601 may comprise any number of deformable portions 602. Moreover, while the deformable portion 602 is shown at the generally flat portion 605, the implant 601 may additionally or alternatively comprise one or more deformable portions 602 at the generally curved portion 607. In some embodiments, portions of the implant 601 other than deformable portions 602 may be at least partially composed of generally non-deformable structures. For example, the implant 601 may be at least partially composed of stainless and/or other materials which may have generally elastic features and/or may be resistant to breaking.

FIGS. 7A-7C illustrate another example implant 701 (e.g., an annuloplasty ring) in accordance with one or more embodiments of the present disclosure. The implant 701 shown in FIGS. 7A-7C has a continuous (e.g., “O-shaped” and/or ovular) structure, in which the implant 701 comprises two generally flat portions 705 including a first generally flat portion 705a and/or a second generally flat portion 705b, which may extends on either side into a generally curved portion 707 configured to form a continuous loop.

The implant 701 may comprise one or more deformable portions 702, which can include a first deformable portion 702a and/or a second deformable portion 702b. For illustrative purposes, a covering 706 is not shown over the deformable portions 702 in FIGS. 7A-7C to show an example position and/or structure of the deformable portion 702. However, a covering 706 of the implant 701 may be configured to at least partially cover the deformable portions 702 and/or other portions of the implant 701. In some embodiments, the deformable portions 702 may be at least partially composed of one or more generally flexible and/or breakable materials. For example, the deformable portions 702 may be at least partially composed of ceramic and/or other breakable materials. As shown in FIGS. 7A-7C, the first deformable portion 702a may be situated across and/or opposite from the second deformable portion 702b. In other words, a line drawn between the first deformable portion 702a and the second deformable portion 702b may bisect the implant 701.

In some embodiments, the one or more deformable portions 702 may be at least partially composed of materials configured to break to create a disconnect and/or separation between some portions of the implant. As shown in FIG. 7B, a covering 706 may be configured to extend over disconnected portions of the implant 701. For example, breaking of the deformable portion 702 may not be configured to cause a break and/or tear in the covering 706 over the deformable portion 702. In some embodiments, the covering 706 may advantageously be configured to retain fragments and/or pieces of a broken deformable portion 702 and/or to prevent such fragments and/or pieces from contacting tissue and/or entering a blood stream. The covering 706 may be at least partially composed of cloth, polyurethane, and/or any other material suitable for contact with human tissue.

In some embodiments, one or more deformable portions 702 may be configured to at least partially bend in response to expansion forces at the implant 701 (e.g., created by a balloon and/or mechanical expansion device). The deformable portions 702 may be composed at least partially of non-elastic materials such that the deformable portion 702 may be configured to cause permanent deformation of the implant 701 in response to expansion forces. In this way, the implant 701 may be configured to be minimally restrictive to other devices (e.g., replacement valves) delivered during future procedures.

As shown in FIG. 7B, both the first deformable portion 702a and/or the second deformable portion 702b may be at least partially composed of breakable materials configured to break in response to expansion forces. In this way, the implant 701 may be configured to be broken into two disconnected body portions, including a first body portion 701a and a second body portion 701b. By forming separate portions, restrictive forces of the implant 701 may advantageously be minimized and/or negated following expansion of the implant 701. In some embodiments, the first body portion 701a and the second body portion 701b may be approximately equal in size and/or shape. For example, the first deformable portion 702a may be situated across from the second deformable portion 702b such that the first deformable portion 702a and the second deformable portion 702b bisect the implant 701 into generally equally sized body portions.

As shown in FIG. 7C, one or more of the deformable portions 702 may be at least partially composed of a generally flexible material. For example, the first deformable portion 702a may be composed of a breakable material and/or the second deformable portion 702b may be composed of generally flexible materials. In this example, after the first deformable portion 702a breaks, the second deformable portion 702b may be configured to allow a separation and/or gap in the first deformable portion 702a to be widened as needed to create needed space for a replacement and/or supplemental device. The second deformable portion 702b may have generally non-elastic properties such that the second deformable portion 702b may not be configured to return to the form shown in FIG. 7A following deformation.

The deformable portions 702 may be configured to be situated on generally opposite and/or facing sides of the implant 701. In this way, the deformable portions 702 may be configured to minimize restriction forces of the implant 701 following deformation. For example, as shown in FIG. 7C, the second deformable portion 702b may be configured to form a hinge for an opening created in the first deformable portion 702a. Because the second deformable portion 702b is situated opposite the first deformable portion 702a, the second deformable portion 702b may be configured to allow maximal widening of the opening in the first deformable portion 702a.

While two deformable portions 702 are shown in FIGS. 7A-7C, the implant 701 may comprise any number of deformable portions 702. Moreover, while the deformable portions 702 are shown at the generally flat portions 705, the implant 701 may additionally or alternatively comprise one or more deformable portions 702 at the generally curved portion 707. While a breakable portion (e.g., the first deformable portion 702a) and a flexible portion (e.g., the second deformable portion 702b) are shown in combination in the implant 701 of FIG. 7C, such portions may be implemented independently and/or in combination.

FIGS. 8A-8C illustrate another example implant 801 (e.g., an annuloplasty ring) in accordance with one or more embodiments of the present disclosure. The implant 801 shown in FIGS. 8A-8C has a non-continuous and/or open (e.g., “C-shaped”) structure forming a partial ring, in which the implant 801 comprises a first end portion 803a and a second end portion 803b, with a gap and/or separation separating the first end portion 803a and the second end portion 803b. The implant 801 may have a generally curved structure and/or may comprise one or more generally flat portions (e.g., at or near a deformable portion 802).

The implant 801 may comprise one or more deformable portions 802. For illustrative purposes, a covering 806 is not shown over the deformable portions 802 in FIGS. 8A-8C to show an example position and/or structure of the deformable portion 802. However, a covering 806 of the implant 801 may be configured to at least partially cover the deformable portions 802 and/or other portions of the implant 801. In some embodiments, the deformable portions 802 may be at least partially composed of one or more generally flexible and/or breakable materials. For example, the deformable portions 802 may be at least partially composed of one or more flexible materials to allow widening of the gap between the first end portion 803a and the second end portion 803b.

In some embodiments, the one or more deformable portions 802 may be at least partially composed of materials configured to break to create a disconnect and/or separation between some portions of the implant. As shown in FIG. 8B, a covering 806 may be configured to extend over disconnected portions of the implant 801. For example, breaking of the deformable portion 802 may not be configured to cause a break and/or tear in the covering 806 over the deformable portion 802. In some embodiments, the covering 806 may advantageously be configured to retain fragments and/or pieces of a broken deformable portion 802 and/or to prevent such fragments and/or pieces from contacting tissue and/or entering a blood stream. The covering 806 may be at least partially composed of cloth, polyurethane, and/or any other material suitable for contact with human tissue.

In some embodiments, one or more deformable portions 802 may be configured to at least partially bend in response to expansion forces at the implant 801 (e.g., created by a balloon and/or mechanical expansion device). The deformable portions 802 may be composed at least partially of non-elastic materials such that the deformable portion 802 may be configured to cause permanent deformation of the implant 801 in response to expansion forces. In this way, the implant 801 may be configured to be minimally restrictive to other devices (e.g., replacement valves) delivered during future procedures.

As shown in FIG. 8B, the deformable portion 802 may be at least partially composed of breakable materials configured to break in response to expansion forces. In this way, the implant 801 may be configured to be broken into two disconnected body portions, including a first body portion 801a and a second body portion 801b. By forming separate portions, restrictive forces of the implant 801 may advantageously be minimized and/or negated following expansion of the implant 801.

As shown in FIG. 8C, one or more of the deformable portions 802 may be at least partially composed of a generally flexible material. For example, the deformable portion 802 may be at least partially composed of generally flexible materials. In this example, the deformable portion 802 may be configured to allow a gap between the first end 803a and the second end 803b to be widened as needed to create needed space for a replacement and/or supplemental device. The deformable portion 802 may have generally non-elastic properties such that the deformable portion 802 may not be configured to return to the form shown in FIG. 8A following deformation.

The deformable portion 802 may be configured to be situated on generally opposite of the gap between the first end 803a and the second end 803b. In this way, the deformable portion 802 may be configured to minimize restriction forces of the implant 801 following deformation. For example, as shown in FIG. 8C, the deformable portion 802 may be configured to form a hinge for the gap between the first end 803a and the second end 803b. Because the deformable portion 802 is situated opposite and/or across from the gap, the deformable portion 802 may be configured to allow maximal widening of the gap.

While a single deformable portion 802 is shown in FIGS. 8A-8C, the implant 801 may comprise any number of deformable portions 802. Moreover, while the deformable portion 802 is shown opposite the gap between the first end 803a and the second end 803b, the implant 801 may additionally or alternatively comprise one or more deformable portions 802 at other points of the implant 801. The implant in FIG. 8C may comprise a covering configured to at least partially cover the first portion 801a, the second portion 801b and/or the deformable portion 802.

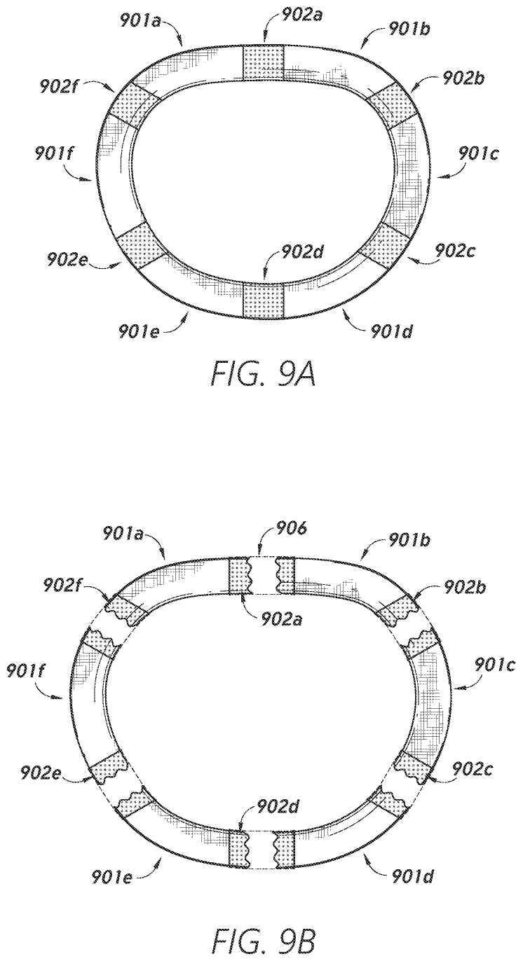

FIGS. 9A and 9B illustrate example implants comprising multiple deformable portions 902, in accordance with one or more embodiments of the present disclosure. Implants described herein may comprise any number of deformable portions 902. As shown in FIGS. 9A and 9B, an implant may comprise six deformable portions, including a first deformable portion 902a, a second deformable portion 902b, a third deformable portion 902c, a fourth deformable portion 902d, a fifth deformable portion 902e, and a sixth deformable portion 902f. One or more of the deformable portions 902 may be at least partially composed of breakable materials, as shown in FIG. 9B. When the one or more deformable portions 902 break, the implant may be configured to be broken into six disconnected body segments, including a first segment 901a, a second segment 901b, a third segment 901c, a fourth segment 901d, a fifth segment 901e, and a sixth segment 901f. In some embodiments, the implant may comprise a covering 906 configured to at least partially enclose the implant and/or to cover any gaps formed between the segments 901 of the implant.

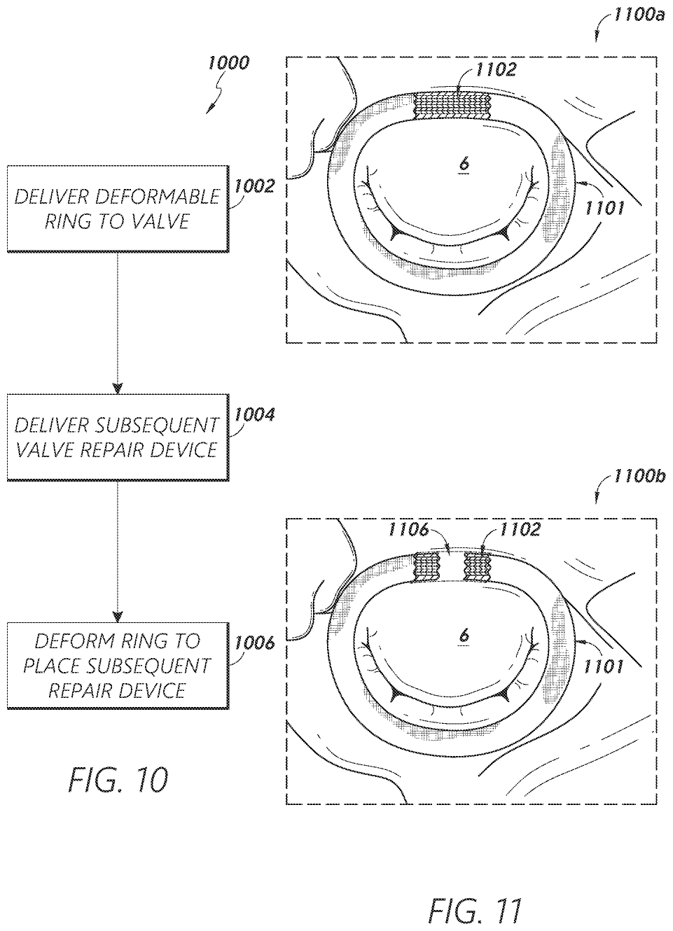

FIG. 10 is a flowchart illustrating steps of a process 1000 for delivering and/or deforming a valve repair device in accordance with one or more embodiments of the present disclosure. FIG. 11 provides certain images depicting various features associated with steps of the process moo depicted in FIG. 10.

At step 1002, the process 1000 involves delivering (e.g., via a catheter) a deformable implant 1101 (e.g., an annuloplasty ring) to a valve 6 of a heart (e.g., a mitral valve) at the annulus and/or other portion of the valve 6, as shown in image 1100a of FIG. 11. In some examples, a delivery (e.g., annuloplasty) procedure includes a surgical procedure where the patient's chest is cut open to access the heart of the patient (e.g., the heart is visible to the physician). In such procedures, the patient's heart is generally stopped and the patient is connected to a cardiopulmonary bypass machine (also referred to as “a heart-lung machine”) (not illustrated) that is configured to take over the function of the patient's heart and lungs. Such procedures are often referred to as “on-pump” or “open heart” procedures. In one example, the patient's heart is stopped, and the physician accessed the mitral valve through the left atrium. Although various annuloplasty procedures are discussed herein in the context of an open-heart surgery, the procedures can be implemented in other manners, such as a minimally invasive procedure where the heart is accessed through a small incision, an off-pump procedure where the heart is still beating and not connected to a heart-lung machine, and so on. In one example of a minimally invasive procedure, an endoscope or another medical instrument can the inserted through a small incision in the patient and navigated to access the target anatomy for the surgery. Further, in some instances, the techniques discussed herein can be applicable to other types of medical procedures, such as a resection procedure (e.g., removing tissue from enlarged or misshapen leaflets), commissurotomy procedure (e.g., cutting the valve commissures to separate the valve leaflets), chordae tendineae procedure, and procedure to decalcify valve/annulus tissue, or any other procedure relating to a heart valve or other anatomy.

In some cases, the implant 1101 may be delivered while a patient is at a relatively young age (e.g., pre-adult). The patient may require additional procedures later in life, which may require removing and/or breaking the implant 1101. Accordingly, the implant 1101 may comprise one or more deformable portions 1102 configured to allow surgeons to permanently deform the implant 1101 to facilitate replacement of the implant 1101 and/or addition of a supplemental device (e.g., a replacement heart valve).

At step 1004, the process 1000 involves delivering (e.g., via a catheter) a subsequent valve repair device (not shown) to the valve 6. The subsequent valve repair device and/or delivery systems associated with delivery of the subsequent valve repair device may be configured to apply expansion force to the implant 1101. At step 1006, the process moo involves deforming the implant 1101 to accommodate the subsequent valve repair device, as shown in image 1100b of FIG. 11, in which the deformable portion 1102 may be at least partially composed of breakable materials to allow the deformable portion 1102 to break in response to the expansion forces. In some embodiments, the implant 1101 may comprise a covering 1106 configured to at least partially enclose the implant 1101 and/or to extend over an opening created in the deformable portion 1102. The covering 1106 may be configured to stretch to some extent to allow a circumference and/or other area of the implant 1101 to be expanded.

Additional Features and Examples

The above description of examples of the disclosure is not intended to be exhaustive or to limit the disclosure to the precise form disclosed above. While specific examples, and examples, are described above for illustrative purposes, various equivalent modifications are possible within the scope of the disclosure, as those skilled in the relevant art will recognize. For example, while processes or blocks are presented in a given order, alternative examples can perform routines having steps, or employ systems having blocks, in a different order, and some processes or blocks may be deleted, moved, added, subdivided, combined, and/or modified. Each of these processes or blocks can be implemented in a variety of different ways. Also, while processes or blocks are at times shown as being performed in series, these processes or blocks can instead be performed in parallel or can be performed at different times.

Certain terms of location are used herein with respect to the various disclosed examples. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

It should be understood that certain ordinal terms (e.g., “first” or “second”) can be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described can be performed in any suitable or desirable temporal order. Furthermore, any of the illustrated and/or described operations or steps can be omitted from any given method or process, and the illustrated/described methods and processes can include additional operations or steps not explicitly illustrated or described.

It should be appreciated that in the above description of examples, various features are sometimes grouped together in a single example, figure, or description thereof for the purpose of streamlining the disclosure and aiding in the understanding of one or more of the various aspects of the disclosure. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the disclosure should not be limited by the particular examples described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, refers to two or more elements that can be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number can also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provide an industry-accepted tolerance for its corresponding term and/or relativity between items. For some industries, an industry-accepted tolerance is less than one percent, while for other industries, the industry-accepted tolerance can be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances can be more or less than a percentage level (e.g., dimension tolerance of less than approximately ±1%). Some relativity between items can range from a difference of less than a percentage level to a few percent. Other relativity between items can range from a difference of a few percent to magnitude of differences.

One or more examples have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks can also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more examples are used herein to illustrate one or more aspects, one or more features, one or more concepts, and/or one or more examples. A physical example of an apparatus, an article of manufacture, a machine, and/or of a process can include one or more of the aspects, features, concepts, examples, etc. described with reference to one or more of the examples discussed herein. Further, from figure to figure, the examples can incorporate the same or similarly named functions, steps, modules, etc. that can use the same, related, or unrelated reference numbers. The relevant features, elements, functions, operations, modules, etc. can be the same or similar functions or can be unrelated.

What is claimed is:

1. A device for treating a heart valve comprising:
a first body portion;
a second body portion; and
a first deformable portion situated between the first body portion and the second body portion;
wherein the first body portion, the second body portion, and the first deformable portion are configured to be attached to an annulus portion of the heart valve.

2. The device of claim 1, further comprising an outer covering configured to surround the first body portion, the second body portion, and the first deformable portion.

3. The device of claim 1, wherein the first deformable portion is configured to break in response to expansion forces.

4. The device of claim 1, wherein the first deformable portion is configured to bend in response to expansion forces.

5. The device of claim 1, wherein the first body portion, the second body portion, and the first deformable portion are configured to form a partial ring with a gap between the first body portion and the second body portion.

6. The device of claim 1, wherein the first body portion, the second body portion, and the first deformable portion are configured to form a continuous ring.

7. The device of claim 1, further comprising a second deformable portion situated between the first body portion and the second body portion and across from the first deformable portion.

8. The device of claim 7, wherein the first body portion, the second body portion, the first deformable portion, and the second deformable portion are configured to form a continuous ring in a generally circular shape.

9. The device of claim 7, wherein the first body portion, the second body portion, the first deformable portion, and the second deformable portion are configured to form a continuous ring having a first generally flat portion.

10. The device of claim 9, wherein the first deformable portion is situated at the first generally flat portion.

11. The device of claim 1, further comprising:
a third body portion;
a second deformable portion situated between the second body portion and the third body portion; and
a third deformable portion situated between the first body portion and the third body portion.

12. A method for treating a heart valve comprising:
delivering a deformable device to an annulus of the heart valve, the deformable device comprising:
a first body portion;
a second body portion; and
a first deformable portion situated between the first body portion and the second body portion;
wherein the first body portion, the second body portion, and the first deformable portion are configured to be attached to an annulus portion of the heart valve;
delivering a subsequent repair device to the heart valve;
permanently deforming the deformable device to allow attachments of the subsequent repair device at the heart valve; and
attaching the subsequent repair device at the heart valve.

13. The method of claim 12, wherein the subsequent repair device is a replacement heart valve.

14. The method of claim 12, wherein the deformable device further comprises an outer covering configured to surround the first body portion, the second body portion, and the first deformable portion.

15. The method of claim 12, wherein the first deformable portion is configured to break in response to expansion forces from a medical device.

16. The method of claim 12, wherein the first deformable portion is configured to bend in response to expansion forces from a medical device.

17. The method of claim 12, wherein the first body portion, the second body portion, and the first deformable portion are configured to form a non-continuous ring with a gap between the first body portion and the second body portion.

18. The method of claim 12, wherein the first body portion, the second body portion, and the first deformable portion are configured to form a continuous ring.

19. The method of claim 12, further comprising a second deformable portion situated between the first body portion and the second body portion and across from the first deformable portion.

20. The method of claim 12, wherein the deformable device further comprises:
a third body portion;
a second deformable portion situated between the second body portion and the third body portion; and
a third deformable portion situated between the first body portion and the third body portion.
